(19) United States
(12) Patent Application Publication
(10) Pub. No.: US 2012/0123171 A1
(43) Pub. Date: May 17, 2012

(54) Catalytic reduction of lignin acids and substituted aliphatic carboxylic acid compounds

(75) Inventor: Melvin Keith Carter, Lincoln, CA (US)

(73) Assignee: Carter Technologies, Lincoln, CA (US)

(21) Appl. No.: 12/927,400

(22) Filed: Nov. 15, 2010

(51) Int. Cl. C07C 37/00; C07C 29/149; C07C 1/207; C07C 41/18

(52) U.S. Cl. 568/651; 568/799; 568/652; 568/885; 585/9; 585/400

(57) ABSTRACT

Renewable resources comprising bagasse, corn stover, wood sawdust and switch grass are subject to direct catalytic conversion or bio-fermentation processes producing ethanol and organic by products leaving complex lignin compounds as waste for disposal. Chemical conversion of lignin compounds to aromatic lignin acids followed by reductive hydrogenation to cresol and substituted creosol compounds prepares these natural resources for chemical conversion to a form of gasoline and valued industrial compounds. The process disclosed herein is also applicable to organic carboxylic acid compounds such as natural oils producing valued liquid hydrocarbon fuels.

Specifically catalytic reactions are taught for reductive chemical hydrogenation of lignin acids comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid and substituted aliphatic carboxylic acid comprising citric and oleic acid compounds in contact with an iron or steel metal surface, a promoter comprising an alkali metal sulfate and a catalyst comprising Co(II)—Co(III) or Mn(II)—Co(III) compound using hydrogen gas at ambient to 10 atmospheres pressure. This process readily forms valued organic compounds from waste natural materials thereby increasing their value.

### REFERENCES CITED

U.S. Patent Documents

| Patent No. | Issue Date | Author | Comments |
|---|---|---|---|
| 7,126,024 | Oct. 24, 2006 | D A Morgenstern, | Catalytic conversion of alkaline alcohols to carboxylic acid |
| | | J P Coleman, | salts using a Fe/Ni/Cu dehydrogenation catalyst. |
| | | J M Allman | |
| 5,268,509 | Dec. 7, 1993 | O Immel, D Liebsch, | Catalytic hydrogenation of nitriles to form primary amines |
| | | H-H Schwarz, S Wendel, | using an iron catalyst with ammonia at 80 C. to 180 C. and |
| | | P Fischer | 20 to 400 atmospheres pressure. |
| 4,994,428 | Feb. 19, 1991 | W K Bell, W O Haag | Fischer-Tropsch conversion of wet syngas to liquid |
| | | | hydrocarbons on a promoted iron catalyst at 160 C. to 350 C. |
| 4,532,209 | Jul. 30, 1985 | S Hagedorn | Cresol from chemical conversion in acidic medium of |
| | | | 4-methylcyclohexa-3,5-diene-1,2-diol-1-carboxylic acid. |
| 4,465,872 | Aug. 14, 1984 | T Suzuki, S Hashimoto, | Peroxide oxidation of p-tolualdehyde to p-cresol in |
| | | M Orisaku & R Nakno | aqueous formic acid at 50 C. to 150 C. |
| 4,301,308 | Nov. 17, 1981 | R Canavesi, F Ligorati, | o-Cresol is prepared from gaseous methanol + phenol at |
| | | & G Aglietti | 200 C. to 400 C. over alumina particles. |

### BACKGROUND

1. Field of Invention

Renewable resources including bagasse, corn stover, wood sawdust, switch grass, recycled cellulose and starch materials are subject to direct catalytic conversion or bio-fermentation processes producing ethanol and organic by products leaving complex lignin compounds as waste for disposal. Chemical conversion of lignin compounds to aromatic lignin acids followed by reductive hydrogenation to cresol and substituted creosol compounds prepares these natural resources for chemical conversion to a form of gasoline and industrial compounds. The process disclosed herein is also applicable to organic carboxylic acid compounds such as natural oils producing valued organic products and hydrocarbon fuels.

Catalytic reactions are taught for reductive chemical hydrogenation of lignin acids comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid and substituted aliphatic carboxylic acid comprising citric and oleic acid compounds in contact with an iron or steel metal catalyst, a promoter comprising an alkali metal sulfate and a catalyst comprising Co(II)—Co(III), Mn(II)—Co(III) or V(II)—Co(III) compound using hydrogen gas at ambient to 10 atmospheres pressure.

2. Description of Prior Art

The chemical process industry has grown to maturity based on petroleum feed stocks. Petroleum is a non-renewable resource that may become unavailable in the next 100 years. This planet Earth fosters continual growth of numerous carbohydrate based plants including fruits, vegetables and grain food sources plus their supporting plant stalks and related cellulose materials. Grains, corn cobs, the support plant stalks and certain grasses are subject to direct catalytic conversion and bio-fermentation processes producing ethanol and organic by products leaving complex lignin compounds as waste for disposal. Chemical conversion of lignin compounds to aromatic lignin acids followed by reductive hydrogenation to cresol and substituted creosol compounds prepares these natural resources for chemical conversion to a form of gasoline. A major industry is blooming in ethanol production but the published conversion efficiencies based on total cellulose starting material are low. These conversion efficiencies can be improved substantially by complete utilization of waste lignins. Ethanol is becoming more available as a renewable resource and this application teaches catalytic hydrogenation of lignin acids and non-lignin acids to valued cresols, substituted creosols and related hydrocarbons in preparation for production of a form of gasoline and chemical intermediates for use in the chemical process industry.

Prior art discloses conversion of chemical compounds derived from petroleum processes to cresols by oxidation, reactive combination or reactive ring closure but none of these reactions teach conversion of lignin acids or non-lignin acid organic compounds to cresols or aliphatic hydrocarbons respectively. U.S. Pat. No. 4,301,308, issued Nov. 17, 1981, introduced a process for preparation of o-cresol by reacting methanol with vaporized phenol at temperatures in the range of 200° C. to 400° C. over alumina particles. U.S. Pat. No. 4,465,872, issued Aug. 14, 1984, teaches a process for peroxide chemical oxidation of p-tolualdehyde to p-cresol in aqueous formic acid at temperatures in the range of 50° C. to 150° C. U.S. Pat. No. 4,532,209, issued Jul. 30, 1985, discloses a process for a reactive ring closure of 4-methylcyclohexa-3,5-diene-1,2-diol-1-carboxylic acid to cresol in an acidic medium.

Iron materials have been employed in chemical conversion processes at times as a co-reactant to consume oxygen byproducts and as catalysts. Catalytic chemical conversion of alkaline alcohols, alcohol amines or alcohols in the presence of amines, to carboxylic acid salts using a Fe/Ni/Cu dehydrogenation catalyst as taught in U.S. Pat. No. 7,126,024, issued Oct. 24, 2006. This is chemically similar to an oxidation reaction. Nitrile compounds have been reduced to amines with hydrogen and ammonia gases on an iron catalyst at 80° C. to 180° C. and 20 to 400 atmospheres pressure as disclosed in U.S. Pat. No. 5,268,509, issued Dec. 7, 1993. Iron has been employed as the primary reaction conversion catalyst for Fischer-Tropsch reactions. For example, chemical conversion of wet syngas to hydrocarbons containing liquids has been conducted on a promoted iron catalyst at 160° C. to 350° C. in U.S. Pat. No. 4,994,428, issued Feb. 19, 1991. While these are all productive uses of iron catalysts none of these disclosures teach use of iron or steel catalysts for chemical reduction of carboxylic acids to methyl substituted compounds as cresols, substituted creosols, alcohols or hydrocarbon compounds.

The above reported chemical processes have been conducted using available petroleum derived chemical compounds and are, therefore, distinctly different from catalytic reductive hydrogenation of renewable resources, specifically lignin acid compounds, to valued cresol and substituted creosol products. The process disclosed herein is also applicable to organic carboxylic acid compounds known as natural fats and oils producing valued liquid hydrocarbon fuels.

### SUMMARY OF THE INVENTION

This invention describes chemical methods using selected transition metal catalysts for reductive hydrogenation of lignin acids and non-lignin acid organic carboxylic acid compounds to cresols, substituted creosols and hydrocarbon products. This process has been shown to be effective for reductive conversion of lignin acids comprising 3,4-dihydroxy-5-methoxybenzoic acid, 3-hydroxy-4-methoxybenzoic acid and 4-hydroxybenzoic acid as well as for aliphatic carboxylic acid compounds comprising oleic acid over zero valent transition metals comprising iron and steel to cresols, substituted creosols and aliphatic hydrocarbons.

It is an object of this invention, therefore, to provide a catalytic process facilitating reductive conversion of lignin acids to cresols and creosols. It is another object of this invention to catalytically reduce non-lignin acid organic carboxylic acid compounds to hydrocarbons. Other objects of this invention will be apparent from the detailed description thereof which follows, and from the claims.

### DETAILED DESCRIPTION OF THE INVENTION

Catalytic hydrogenation of aromatic lignin acids to cresol, creosol and substituted creosol compounds prepares these valuable derivatives of natural resources for chemical conversion to a form of gasoline and valued industrial compounds. The process is also applicable to aliphatic carboxylic acid compounds such as natural oils producing valued liquid hydrocarbon fuels. Specifically catalytic reactions are taught for reductive chemical hydrogenation of lignin acids comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid to cresol, creosol and substituted creosols, and substituted aliphatic carboxylic acid comprising citric and oleic acid compounds are reduced to hexanol and C₁₈ hydrocarbons respectively. These reductions take place with lignin acids or aliphatic carboxylic acid compounds in contact with an iron or steel metal surface, a promoter comprising an alkali metal sulfate and a catalyst comprising Co(II)—Co(III) or Mn(II)—Co(III) using hydrogen gas at ambient to 10 atmospheres pressure.

This process employs transition metal catalysts for which the transition metals and directly attached atoms possess C₄ᵥ, D₄ₕ or D_{2d} point group symmetry. The catalysts have been designed based on a formal theory of catalysis, and the catalysts have been produced, and tested without pre-conditioning to prove their activity as prepared. The theory of catalysis rests upon a requirement that a catalyst possess a molecular string such that transitions from one molecular electronic configuration to another be barrier free so reactants may proceed freely to products as driven by thermodynamic considerations. Catalysts effective for stated chemical conversions to products can be made from bi-metal, tri-metal and/or poly-metal backbone or molecular string type compounds of mixed valence form the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof. These catalysts are made in the absence of oxygen so as to produce compounds wherein the oxidation state of the transition metal is low, typically divalent and trivalent metals. Mixed transition metal compounds have also been found to be effective catalysts for non-oxidative chemical conversions.

Iron and steel surfaces are the sites of hydrogenation but a promoter and a catalyst are required to enable the reductive chemistry. It is believed that the catalyst assists in bond opening and the promoter functions to assist in hydrogenation of the metallic surface. It is also apparent that water vapor, a byproduct of the reduction reaction, inhibits the rate of the reaction. Thus, by instituting a pulsed hydrogen gas flow, reaction products can be swept from the metallic surface with the byproduct water vapor. For example, reduction of 4-hydroxybenzoic acid with a steady gas flow produced approximately 25 percent product while the pulsed flow process produced nearly 100 percent conversion.

Thermodynamic considerations determine which chemical compounds are reduced, however reduction becomes increasingly favored as hydrogen pressure is increased. For example, 4-hydroxybenzoic acid was converted to 13 percent product at ambient hydrogen pressure while the reduction process produced nearly 100 percent product at 30 psig. Similar relative pressure related conversion efficiencies were observed for oleic acid. Thus, reductive chemical conversion of carboxylic acid compounds, activated by the selected catalysts and a promoter on iron or steel surfaces, are taught herein producing methyl substituted analogs of the original compounds.

### Catalyst Preparation

### Example 1

### Preparation of the Co(Ii)—Co(III) and the Mn(II)—Co(III) Catalysts were Conducted in a Short Time Sequence Preferably in an Inert Gas Environment

Glass vial a—To 0.0115 g tetrachlorocatechol add 0.0025 g Na₂CO₃ in 1 g water, heat and stir until dissolved. Immediately add 0.0110 g CoCl₂-6H₂O and stir to form product A. Heat at 160° C. for approximately 2 minutes to form product. Glass vial b—To 0.0115 g tetrachlorocatechol add 0.0025 g Na₂CO₃ in 1 g water, heat and stir as before until dissolved. Add 0.0124 g Co(NH₃)₆Cl₃ and stir to form the product. Heat the vial at 160° C. for approximately 2 minutes to form product. Mix product a and product b together, add an additional 1 g water and add 0.0115 g tetrachlorocatechol, heat as before and stir until a dark color product forms.

### Example 2

Glass vial a—To 0.0229 g tetrachlorocatechol add 0.0049 g Na₂CO₃ in 1 g water, heat and stir until dissolved. Immediately add 0.0183 g MnCl₂-4H₂O and stir to form product A. Heat at 160° C. for approximately 2 minutes. Glass vial b—To 0.0229 g tetrachlorocatechol add 0.0049 g Na₂CO₃ in 1 g water, heat and stir as before until dissolved. Add 0.0247 g Co(6NH₃)Cl₃ and stir to form the product. Mix products a and b together, add an additional 1 g water and add 0.0229 g tetrachlorocatechol, heat as before and stir until a dark color product forms.

### Examples of Catalytic Chemical Conversion

Specific examples of the conditions of catalytic reductive chemical conversion to products are provided here.

### Example A

### p-Cresol Formation

The reaction equipment consisted of a 250 mL three neck round bottom pyrex glass flask fit with a thermocouple, a one eighth inch diameter stainless steel line for hydrogen gas introduction, a one quarter inch line for product vapor removal in series with a gas vent line. The reactor was wrapped with a thick layer of fiber mat insulation to maintain a uniform temperature throughout the reaction chamber. Two pieces of carbon steel, each 2″×¾″×0.032″ were placed in the bottom of the flask. The reactants, 4.0 g of

4-hydroxy benzoic acid plus 0.022 g Co(II, III) tetrachlorocatechol catalyst plus 0.405 g Na₂SO₄, were ground together in a mortar and pestle and placed in the flask on top of the steel strips. Hydrogen gas was introduced into the bottom of the flask at a flow rate of 10 mL/minute to flush air from the reactor. After flushing the reactor was heated to 285° C. to 288° C. for a period of one hour with ambient pressure hydrogen gas flowing to form 0.41 gram (13 percent) p-cresol product (verified by boiling point).

### Example B

### p-Cresol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 3.246 g of 4-hydroxy benzoic acid plus 0.0108 g Co(II, III) tetrachlorocatechol catalyst plus 0.304 g Na₂SO₄, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 288° C. to 290° C. for a period of three hours and forty minutes. The reactor was flushed with a short burst of hydrogen, by sharp pressure drops followed by re-pressurization, every 5 to 10 minutes to sweep out water vapor. Once the reactor was cool it was opened and 2.301 g (95.7%) crude liquid p-cresol was recovered.

### Example C

### Methoxy Cresol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 2.853 g of 4-hydroxy-3-methoxybenzoic acid plus 0.0158 g Co(II, III) tetrachlorocatechol catalyst plus 0.315 g Na₂SO₄, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 315° C. to 330° C. for a period of two hours and fifteen minutes. The reactor was flushed with a short burst of hydrogen, by sharp pressure drops followed by re-pressurization, every 5 to 10 minutes to sweep out water vapor. Once the reactor was cool it was opened and 1.31 g (57%) crude liquid methoxy cresol was recovered.

### Example D

### Dimethoxy Cresol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 3.013 g of syringic acid plus 0.0120 g Co(II, III) tetrachlorocatechol catalyst plus 0.356 g Na₂SO₄, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 320° C. to 345° C. for a period of two hours and fifteen minutes. The reactor was flushed with a short burst of hydrogen, by sharp pressure drops followed by re-pressurization, every 5 to 10 minutes to sweep out water vapor. Once the reactor was cool it was opened and 1.334 g (53%) crude liquid dimethoxy cresol was recovered.

### Example E

### Hexanol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 3.136 g of citric acid plus 0.0316 g Co(II, III) tetrachlorocatechol catalyst plus 0.377 g Na₂SO₄, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 228° C. to 249° C. for a period of two hours. The reactor was flushed with a short burst of hydrogen, by sharp pressure drops followed by re-pressurization, every 5 to 10 minutes to sweep out water vapor. Once the reactor was cool it was opened and 0.644 g (39.5%) crude hexanol was recovered.

### Example F

### A C₁₈ Wax

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 5.0 g oleic acid liquid with 0.053 g Mn(II)—Co(III) tetrachlorocatechol catalyst plus 0.52 g Na₂SO₄, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 228° C. to 249° C. for a period of two hours. The reactor was flushed with a short burst of hydrogen, by sharp pressure drops followed by re-pressurization, every 5 to 10 minutes to sweep out water vapor. Once the reactor was cool it was opened and 0.13 g brown wax, likely octadecane or octadecene, (10%) was recovered.

What is claimed:

1. Catalytic hydrogenation of substituted carboxylic acid compounds in contact with an iron or steel metal surface, a promoter comprising an alkali metal sulfate and a catalyst comprising Co(II)—Co(III) or Mn(II)—Co(III) compound, made from bi-metal, tri-metal and/or poly-metal backbone or molecular string type compounds of mixed valence produced from the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, using hydrogen gas at ambient to 10 atmospheres pressure forming substituted methyl compounds.

2. Catalytic hydrogenation of substituted lignin acids comprising 4-hydroxy-3,5-dimethoxybenzoic acid, 4,5-dihydroxy-3-methoxybenzoic acid, 4-hydroxy-3-methoxybenzoic acid and 4-hydroxybenzoic acid compounds in contact with an iron or steel metal surface, a promoter comprising an alkali metal sulfate and a catalyst comprising Co(II)—Co(III) or Mn(II)—Co(III) compound, made from bi-metal, tri-metal and/or poly-metal backbone or molecular string type compounds of mixed valence from the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, using hydrogen gas at ambient to 10 atmospheres pressure forming substituted methyl lignin compounds comprising cresols and substituted cresols.

3. Catalytic hydrogenation of substituted aliphatic carboxylic acid compounds comprising citric and oleic acid in contact with an iron or steel metal surface, a promoter comprising an alkali metal sulfate and a catalyst comprising Co(II)—Co(III) or Mn(II)—Co(III) compound, made from bi-metal, tri-metal and/or poly-metal backbone or molecular string type compounds of mixed valence from the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, using hydrogen gas at ambient to 10 atmospheres pressure forming substituted methyl organic compounds comprising hexanol and C₁₈ hydrocarbons respectively.

4. Catalytic hydrogenation of substituted lignin acids comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid and substituted aliphatic carboxylic acid compounds comprising citric and oleic acid in contact with an iron or steel metal surface, a promoter comprising an alkali metal sulfate and a catalyst comprising Co(II)—Co(III) or Mn(II)—Co(III) compound, made from bi-metal, tri-metal and/or poly-metal backbone or molecular string type compounds of mixed valence from the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, using hydrogen gas at ambient to 10 atmospheres pressure forming substituted methyl lignin compounds comprising cresols, substituted cresols, hexanol and C₁₈ hydrocarbon compounds respectively.

5. Catalytic hydrogenation of 4-hydroxybenzoic acid, 4-hydroxy-3-methoxybenzoic acid, syringic acid (4-hydroxy-3,5-dimethoxybenzoic acid) and 4,5-dihydroxy-3-methoxybenzoic acid in contact with an iron or steel metal surface, a promoter comprising an alkali metal sulfate and a catalyst comprising Co(II)—Co(III) or Mn(II)—Co(III) compound, made from bi-metal, tri-metal and/or poly-metal backbone or molecular string type compounds of mixed valence from the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, using hydrogen gas at ambient to 10 atmospheres pressure forming p-cresol, methoxy cresol, dimethoxy cresol and dihydroxy cresol respectively.

6. Catalytic hydrogenation of citric and oleic acid in contact with an iron or steel metal surface, a promoter comprising an alkali metal sulfate and a catalyst comprising Co(II)—Co(III) or Mn(II)—Co(III) compound, made from bi-metal, tri-metal and/or poly-metal backbone or molecular string type compounds of mixed valence from the transition metals comprising titanium, vanadium, chromium, manganese, iron, cobalt, nickel, copper, zirconium, niobium, molybdenum, ruthenium, rhodium, palladium, silver, hafnium, tantalum, tungsten, rhenium, osmium, iridium, platinum, gold or combinations thereof, using hydrogen gas at ambient to 10 atmospheres pressure forming hexanol and C₁₈ hydrocarbon compounds respectively.
